(19) United States
(12) Patent Application Publication
(10) Pub. No.: US 2004/0192599 A1
(43) Pub. Date: Sep. 30, 2004

(54) Gene therapy for hemophilia a

(76) Inventors: Andre C Schuh, Toronto (CA); Edward M Conway, Overijise (BE)

Correspondence Address: FISH & RICHARDSON PC, 225 FRANKLIN ST, BOSTON, MA, 02110, US

(21) Appl. No.: 10/480,887

(22) PCT Filed: Jun. 17, 2002
(86) PCT No.: PCT/CA02/00903

Related U.S. Application Data
(60) Provisional application No. 60/298,121, filed on Jun. 15, 2001.

(51) Int. Cl. A61K 38/37; A61K 48/00; C07H 21/04

(52) U.S. Cl. 514/12; 514/44; 530/383; 536/23.5

(57) ABSTRACT

Nucleic acid constructs comprising procoagulant gene sequences under the control of a megakaryocyte-specific promoter are provided. The sequences preferably also comprise a secretory granule sorting domain Also provided are vectors comprising the sequences and methods of gene therapy comprising the use of the various constructs.

### FIELD OF THE INVENTION

The present invention is directed to gene therapy for the treatment of hemophilia A, particularly to gene therapy that is targeted to megakaryocytes and platelets.

### BACKGROUND OF THE INVENTION

Throughout this application, various references are cited to describe more fully the state of the art to which this invention pertains. Full bibliographic information for each citation is found at the end of the specification, immediately preceding the claims. The disclosures of these references are hereby incorporated by reference into the present disclosure. Hemophilia A is an X-linked bleeding disorder caused by an absence or decreased function of Factor VIII (FVIII), resulting from mutations in the FVIII gene. The incidence of hemophilia A is approximately one in 10,000-5,000 males, and results in bleeding in deep tissues, joints and muscles ¹³. Over 70% of patients with hemophilia A are characterized as having the most severe form of the disease, classified according to hemorrhagic symptoms, which are closely correlated with the plasma level of FVIII. The most severely affected individuals have levels of <1%, while more moderate hemorrhagic symptoms are associated with FVIII levels of 1-5%.

The mainstay of treatment of hemophilia A has been replacement therapy with blood products that contain FVIII. Since the introduction of fractionated blood products, the median life expectancy for patients with severe hemophilia extended from 10-15 years to 60-70 years. With longer survival, prevention of the major cause of morbidity of hemophilia A, joint disease, became the focus of attention¹⁴. It is not surprising that prophylaxis with FVIII concentrates became an accepted therapy, committing affected children to regular infusions of FVIII concentrates¹⁵. This of course, requires long-term venous access, and is associated with a high risk of infection. Management of hemophilia A became further complicated in the 1980s with a dramatic rise in transfusion-associated infections, particularly hepatitis and HIV¹⁶. As a result, recombinant FVIII concentrates were developed, and have, in many practices, superseded immunopurified plasma-derived FVIII preparations. Pharmacokinetic studies have shown that the recombinant products are efficacious with respect to prevention of bleeding. However, there are still major concerns, particularly about convenience of administration and the development of FVIII inhibitors.

Although the exact incidence of development of inhibitors to FVIII is difficult to ascertain, it appears to be in the range of 20% of all patients with severe forms of hemophilia A. Attempts to prevent or address the development of inhibitors have been multifaceted, with variable results. Regimens of infusing huge doses of FVIII over a period of years have been developed for use in some patients with low titer inhibitors, but these are expensive and not reliable. Attempts at immune suppression using combinations of chemotherapeutic agents, intravenous gammaglobulin, and extracorporeal adsorption of IgG on protein A columns, have had some success in non-emergent situations¹⁷. Porcine FVIII is often used, but there is currently a worldwide shortage and concerns about infectivity exist. In addition, repeated administration may lead to the development of anti-porcine FVIII antibodies. Prothrombin complex concentrates (PCC)¹⁸ with “bypassing” activity are associated with a high risk of transmitting infections. More recently, intravenous administration of recombinant factor VIIa has been utilized in patients with life-threatening bleeds and FVIII inhibitors. However, this agent is only available in Canada on a compassionate basis, it has a very short half-life, and it is expensive^{19,20}. The advent of “second generation” recombinant FVIII concentrates, which lack the central B-domain of FVIII^{21,22} are reported to have higher specific activity and greater stability both in vitro and in vivo. However B-domain deleted FVIII also induces the production of clinically relevant factor VIII inhibitors.

The molecular events surrounding initiation of coagulation have been extensively examined and revised since the original description of the cascade hypothesis of hemostatic system activation. Following vascular injury, tissue factor (TF) is exposed to the circulation and complexes with factor VIIa, which, in turn, serves to activate factors IX and X, in a process sustained through the activation of FVIII, which is carried in the plasma by von Willebrand Factor (vWF), by factor IXa^{1,2}. These events occur predominantly on activated platelets, where assembly of the factor IXa-FVIIIa complex takes place. The coagulation process is further consolidated by activation of factor XI. Tissue factor pathway inhibitor (TFPI) inhibits factor Xa, thereby regulating the ultimate generation of thrombin. This scheme supports the current view that the TF/VIIa pathway of blood clotting is the major physiological mechanism for triggering coagulation, both in health and disease. Furthermore, it is consistent with the observation that patients with deficiencies of FVIII, vWF or factor IX have clinically severe bleeding tendencies. These new insights into the biochemical and molecular mechanisms active in coagulation have led to innovative approaches to treating patients with a variety of inherited bleeding disorders, including hemophilia A.

Tissue factor (TF) is a cell surface, transmembrane, glycoprotein that is expressed by perivascular cells, as well as by activated monocytes/macrophages³⁻⁵. Its extracellular domain constitutes over 80% of the amino acid sequence of the molecule and provides binding sites for factor VIIa⁶. Central to the initiation of clotting is the conversion of factor VII through cleavage of a single arginine-isoleucine bond to its serine protease active form, factor VIIa. Factor Vila binding to TF, an interaction that results in a dramatic enhancement of its protease activity towards factors IX and X⁷, is mediated by a reaction that occurs predominantly on platelets or endothelial cells. For optimal cofactor function, FVIII must be activated proteolytically by thrombin, which results in the generation of an active FVIII heterodimer (FVIIIa), and the release of the apparently functionless (from a coagulation point of view) B-domain^{8,9}.

vWF is synthesized by endothelial cells and by megakaryocytes. It is localized in α-granules of platelets, and the Weibel-Palade bodies of endothelial cells¹⁰. Release of vWF from either platelets or endothelial cells may be induced by a variety of agonists, including thrombin. vWF consists of multimeric forms of a dimer subunit with a molecular weight of approximately 250 kDa (for review⁸). The mature, processed translation product of vWF is a protein of 2050 amino acids. Following a propeptide at the N-terminus, there are two so-called D-domains, followed by 3 A-domains, another D-domain, 3 short B-domains, and finally 2 C-domains.

vWF plays a critical role in promoting coagulation in at least two ways. Firstly, it promotes platelet adhesion to damaged blood vessel endothelium via a variety of receptors, including fibronectin and collagen types III, IV, and V. Secondly, it serves as a carrier for FVIII so that localized bleeding may be abrogated. With respect to the latter, Montgomery and coworkers¹¹ have recently determined that vWF may also play an intracellular chaperone role for FVIII. Using AtT20 cells, a murine pituitary cell line that has been used widely to study vWF intracellular tracking and regulated release, they demonstrated that vWF could alter the intracellular trafficking of FVIII from a constitutive to a regulated secretory pathway, thereby producing an intracellular storage pool of both procoagulant proteins. More recently, the same groups have determined that megakaryocytes can synthesize and store FVIII with vWF in α-granules that can be retained in progeny platelets¹². The present invention utilises gene therapy approaches to provide a more effective, targeted therapeutic strategy for hemophilia A.

For several reasons, hemophilia has been considered a particularly attractive model in which to undertake gene therapy. First, tissue-specific expression is not believed to be essential, as long as the FVIII has access to the plasma and the site of injury. Second, high level and tightly regulated FVIII expression is not required, since patients with FVIII levels of as low as 5% rarely suffer from significant spontaneous bleeding events. Thus, a dramatic phenotypic improvement would be achieved by raising plasma levels from 1% to 5%. Furthermore, supranormal FVIII levels are not known to be detrimental. Finally, excellent small animal models exist in which gene therapy strategies may be evaluated²³⁻²⁶.

Major advances have been made in the development of retroviral vectors encoding B-domain-deleted FVIII cDNA in an attempt to overcome difficulties in both viral titres and levels of FVIII expression^{27,28}. Several attempts at ex vivo delivery of FVIII have met with limited success. The most promising attempt resulted in high-level expression of FVIII in mouse plasma following retrovirus-mediated ex vivo gene transfer into fibroblasts, followed by implantation into the mice within a collagen matrix²⁷. Unfortunately, these experiments were confounded by only transient expression of adequate levels of FVIII. Longer-term expression has been attained by intravenous injection into newborn haemophilic mice of retroviruses expressing high levels of FVIII. This approach, however, suffers the drawback of a high frequency of neutralizing antibodies²⁹. Other transfection approaches have also been attempted but generally resulted in low level, short-term FVIII expression³⁰.

Considerable progress has also been made in the development of adenoviral vector-mediated in vivo gene therapy approaches for the treatment of hemophilia A. Therapeutic levels of FVIII have been sustained in mice for several weeks^{31,32}. However, only short-term functional expression has been attained in hemophilic dogs³³, due in part to the development of anti-FVIII antibodies. A major obstacle to application of adenoviral vectors to the treatment of hemophilia is the invariant loss of expression with time, since the vector remains episomal³⁴. Another drawback is the induction of an immune response directed against the vector backbone that prevents repeated administration^{34,35}.

Other viral gene transfer systems for hemophilia A, including lentivirus³⁶ and adeno-associated virus (AAV)³⁷, non-viral-based treatments are also being investigated³⁸. Although some of these approaches appear promising, they are still at early stages in development.

In conclusion, despite significant advances in the treatment of hemophilia A, there are still many problems associated with current treatments for this disease. These include the inconvenience of FVIII administration and its short-term efficacy, as well as the appearance of anti-FVIII antibodies. Treatments are very expensive and there are concerns about the safety of viral vectors. Thus, there is a real and unmet need for improved treatments.

### SUMMARY OF THE INVENTION

The present invention is directed to a novel gene therapy strategy for the management of hemophilia A.

The present invention provides a system for the targeted expression of a desired nucleic acid sequence in particular cell types such as megakaryocytes and platelets.

According to one embodiment, bone marrow or other cells are transformed or otherwise genetically modified ex vivo and then delivered to a mammalian recipient. Preferably, the mammalian recipient is a human that has a condition amenable to gene replacement therapy.

According to another embodiment, the cells are transformed or otherwise genetically modified in vivo.

In accordance with one aspect of the invention, there is provided a nucleic acid construct comprising all or part of a gene sequence encoding a procoagulant factor operably linked to an effective megakaryocyte/platelet specific regulatory region.

In a preferred embodiment, the nucleic acid sequence further comprises a secretory granule-sorting domain.

In another preferred embodiment the procoagulant fact is Factor VIII.

In another embodiment the procoagulant factor is hepsin.

In yet another preferred embodiment, the megakaryocyte/platelet specific regulatory region is selected from the group consisting of the PF4 promoter, the platelet integrin alpha IIb/GPIIb promoter and other platelet glycoprotein promoters such as the GPVI promoter.

In another embodiment, preferred secretory granule sorting domains include, but are not limited to the cytoplasmic domain of P-selectin and the carboxy-terminal tails of the proprotein convertases PC5A and PC1. The secretory granule-sorting domain is preferably expressed as an in-frame fusion with the procoagulant protein gene sequence.

In another aspect of the invention, there is provided a vector for expression of the nucleic acid construct.

In a preferred embodiment, the vector is a retroviral vector.

In a further aspect of the invention, cells expressing the nucleic acid construct are provided.

In yet another aspect of the invention, an animal expressing the nucleic acids constructs of the invention is provided.

According to another aspect of the invention, a method of treating hemophilia A is provided. The method comprises: introducing into bone marrow, such that it is then expressed in bone marrow-derived megakaryocyte or stem cells, a construct comprising a procoagulant factor encoding DNA sequence and a tissue-specific promoter operably linked to the procoagulant DNA to facilitate expression in said cells.

In a preferred embodiment, expression of the introduced construct occurs such that the procoagulant factor accumulates in platelet α-granules and is released upon platelet activation.

In one embodiment, the construct is introduced into cells ex vivo and the transfected cells are administered to a patient in need of treatment.

The present invention has several advantages. First, this approach targets procoagulant activity not only to areas of vascular injury, but also to those sites in which secondary “rebleeding” occurs. Second, since the targeted protein is sequestered in α-granules and is not released until platelet activation occurs, even low levels of constitutive transgenic protein production will result in high local factor levels at the sites of bleeding. And third, this approach has a number of immunological advantages as well. Evidence gained from cases of acquired von Willebrand's disease, predict that proteins packaged and delivered from α-granules may not incite alloimmunization³⁹. In addition, since bone marrow-mediated antigen exposure is known to be less immunogenic than is parenteral exposure to the same antigen, and may potentially induce antigen-specific tolerance in both naive and pre-immunized hosts as well⁴⁰, targeted FVIII expression will prevent the formation of FVIII inhibitors in previously untreated patients, and may induce tolerance in the setting of pre-existing FVIII antibodies.

### BRIEF DESCRIPTION OF THE DRAWINGS

The invention is described in more detail herein with reference to the drawings, in which:

FIG. 1 illustrates a BDD-FVIII fusion construct;

FIG. 2 is a graph illustrating the results of a FVIII functional chromogenic assay;

FIG. 3 illustrates retroviral vectors for expression of the nucleic acid constructs of the present invention;

FIG. 4 illustrates BDD-FVIII fusion constructs for the generation of transgenic mice;

FIG. 5 illustrates BDD-FVIII fusion constructs linked to a secretory granule-sorting domain;

FIG. 6 illustrates immunofluorescent staining of transgenic megakayrocytes; and

FIG. 7 illustrates the results of an RT-PCR assay.

### DESCRIPTION OF THE PREFERRED EMBODIMENTS

The present invention addresses the need for improved therapies for diseases associated with abnormal gene expression in megakayocytes and platelets. In particular, a therapeutic modality for Hemophilia A is provided which is designed to act specifically at the site of bleeding and at the time of bleeding. Targeted gene therapy is used to direct the expression of FVIII to platelet α-granules, such that coagulation is specifically initiated by regulated FVIII release following platelet activation at sites of vascular injury. The present invention obviates many of the current problems associated with long-term treatment with FVIII concentrates, and overcomes some of the deficiencies of current gene therapy strategies.

There are two basic approaches to gene therapy, i) ex vivo gene therapy and ii) in vivo gene therapy.

In ex vivo gene therapy, cells are removed from a subject and transfected with a desired gene in vitro. The genetically modified cells are expanded and then implanted back into the subject. Various methods of transfecting cells such as by electroporation, calcium phosphate precipitation, liposomes, microparticles, and other methods known to those skilled in the art can be used in the practice of the present invention.

In in vivo gene therapy, the desired gene is introduced into cells of the recipient in vivo. This can be achieved by using a variety of methods known to those skilled in the art. Such methods include but are not limited to, direct injection of DNA into muscle cells and introduction of DNA in a carrier. Delivery of DNA to the vasculature, the lung, the nervous system and various other organs has been reported.

Various transduction processes can be used for the transfer of nucleic acid into a cell using a DNA or RNA virus. In one aspect of the present invention, a retrovirus is used to transfer a nucleic acid into a cell. Exogenous genetic material encoding a desired gene product is contained within the retrovirus and is incorporated into the genome of the transduced cell. The amount of gene product that is provided in situ is regulated by various factors, such as the type of promoter used, the gene copy number in the cell, the number of transduced/transfected cells that are administered, and the level of expression of the desired product. The present invention provides a selection and optimization of factors to deliver a therapeutically effective dose of Factor VIII or other coagulant factor to a site of injury. The expression vector of the present invention preferably includes a selection gene, for example, a neomycin resistance gene, to facilate selection of transfected or transduced cells.

In the present invention, the therapeutic agent, such as Factor VIII is targetted such that it will have easy access to the plasma and site of injury. The present invention is useful to decrease the morbidity and mortality associated with clotting disorders. In addition to the targeting of Factor VIII for the treatment of Hemophilia A, other pathologies associated with a lack of expression of specific factors by platelets and megakaryocytes can also be treated by the targeted gene therapy approaches of the present invention. The selection and optimization of a particular expression vector for expressing a specific gene product in megakaryocytes/platelets is accomplished by inserting the desired gene under the control of a megakaryocyte specific promoter, transfecting or transducing bone marrow cells in vitro; and determining whether the gene product is present in the cultured cells. The vector construct also preferably includes a sequence which targets expression of the desired gene product to the alpha granules of platelets.

In a preferred embodiment, vectors for megakaryocyte cell gene therapy are viruses, more preferably retroviruses. Replication-deficient retroviruses are incapable of making infectious particles. Genetically altered retroviral expression vectors are useful for high-efficiency transduction of genes in cultured cells and are also useful for the efficient transduction of genes into cells in vivo. Standard protocols for the use of retroviruses to transfer genetic material into cells are known to those skilled in the art. For example, a standard protocol can be found in Kriegler, M. Gene Transfer and Expression, A Laboratory Manual, W. H. Freeman Co, New York, (1990) and Murray, E. J., ed. Methods in Molecular Biology, Vol. 7, Humana Press Inc., Clifton, N.J., (1991).

The expression vector may also be in the form of a plasmid, which can be transferred into the target cells using a variety of standard methodologies, such as electroporation, microinjection, calcium or strontium co-precipitation, lipid mediated delivery, cationic liposomes, and other procedures known to those skilled in the art.

The present invention provides various methods for making and using the above-described genetically-modified megakaryocytes. In particular, the invention provides a method for genetically modifying bone marrow cells of a mammalian recipient ex vivo and administering the genetically modified cells to the mammalian recipient. Preferably, autologous cells are used.

The present invention also provides methods in vivo gene therapy. An expression vector carrying a heterologous gene product is injected into a recipient. In particular, the method comprises introducing a targeted expression vector, i.e., a vector which has a cell-specific promoter.

Genetically modified cells expressing a desired gene product are provided. The desired gene product is determined based on the disease and the therapeutic dose is determined based on the condition of the patient, the severity of the condition, as well as the results of clinical studies of the specific therapeutic agent being administered.

The genetically modified cells are typically administered in an acceptable carrier such as saline or other pharmaceutically acceptable excipients. The genetically modified cells of the present invention are administered in a manner such that they have access to the vascular system.

The present invention specifically provides vectors and cells for the targeted expression of FVIII or other procoagulant factors in megakaryocytes and platelets and directed trafficking of those factors to platelet α-granules. The targeted expression proteins accumulate within α-granules, and are therefore available for regulated local release following platelet activation at sites of injury. Thus, in the case of FVIII targeting, high local levels of FVIII are produced specifically at sites of injury.

A novel FVIII gene construct is provided. Factor VIII is initially synthesized as a 2351 amino acid pre-pro-protein containing a 19 amino acid residue leader peptide. The 2322 amino acid secreted form of FVIII is divided into distinct structural domains in the order A1, A2, B, A3, C1, and C2. The B domain extends from Ser741 to Arg1648 inclusive. During synthesis/secretion, pro-FVIII is cleaved by a proprotein convertase at Glu1649, to yield a large fragment encompassing domains A1-B, and a smaller fragment encompassing domains A3-C2. These two fragments associate with each other. This two-chain molecule is inactive, but subsequently becomes activated by thrombin cleavage at Arg740, which liberates the B domain from the heavy chain.

Because of its size (>7kb), transgenic expression of a full-length FVIII cDNA has been problematic. However, as the B domain is not required for FVIII coagulant activity, a variety of groups have explored the use of modified FVIII cDNAs from which the B domain- encoding portions have been removed, as a means of expressing functional FVIII from a smaller cDNA. B domainless FVIII has been produced by two general means. One approach is to express the heavy (domains A1-A2) and light (domains A3-C2) chains separately, either from the same, or from distinct plasmids. Separately synthesized recombinant heavy and light chains will associate spontaneously with each other to reconstitute active FVIII. The more common approach, however, is to express the heavy and light chains from a single mutant cDNA from which all, or a portion of, the B domain-encoding sequences have been deleted. FVIII/vWF interactions are known to be unaffected by deletion of the B-domain²².

In the present invention, a novel cDNA encoding a B-domain-deleted form of human FVIII, which confers high-level FVIII expression is disclosed.

Human FVIII was used to synthesise, by recombinant PCR, a cDNA that encodes FVIII domains A1-A2 (amino acids 1-740) and A3-C2 (amino acids 1649-2351), joined by a linking fragment encompassing the first 20 and the last 18 B domain amino acid residues (residues 741-760 and 1631-1648, respectively. The resultant protein (lacking amino acid residues 761-1630) is secreted normally, and as the processing motif at Glu1649 and the thrombin cleavage site at Arg740 both remain intact, it is fully functional.

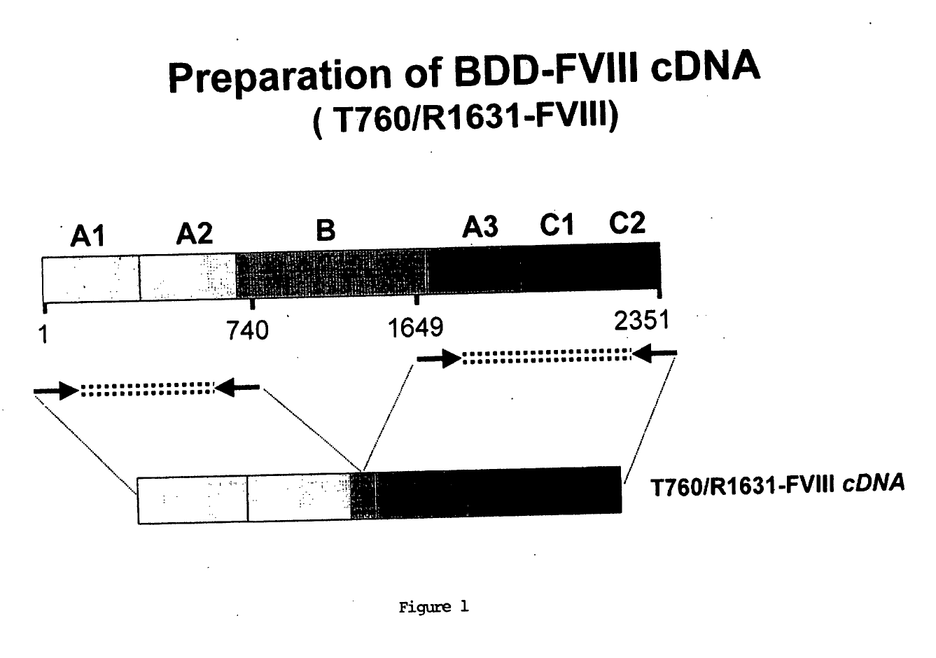

This novel, exemplary BDD-FVIII fusion construct is designated T760/R1631-FVIII cDNA and is illustrated in FIG. 1. It is clearly apparent, however, that other BDD-FVIII constructs can be substituted within the scope of the present invention for targeted expression.

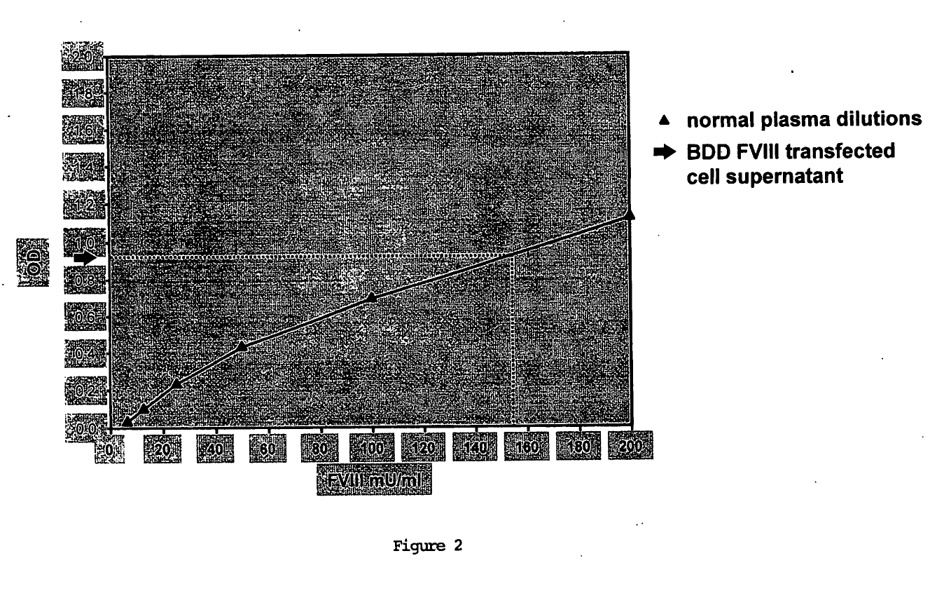

When expressed in COS cells, the T760/R1631-FVIII cDNA construct demonstrated significant FVIII activity as measured using a commercial FVIII procoagulant activity assay (Coamatic [Chromogenic Inc.] The assay measures the cofactor activity of FVIII in FIXa mediated activation of FX. FIG. 2 illustrates the results of one such FVIII functional. chromogenic assay. The standard curve is derived from a commercial source of recombinant FVIII. COS cells transfected with a control vector not including the FVIII construct had an FVIII activity (mU/ml) of 0, while COS cells transfected with a vector expressing the FVIII construct had an activity of >150 mU/ml.

As described above, the function of vWF and FVIII are intimately related. It is well known in the art that the half-life of the non-activated Factor VIII heterodimer strongly depends on the presence of von Willebrand Factor, which exhibits a strong affinity to Factor VIII (yet not to Factor VIIIa) and serves as a carrier protein. It is also known that patients suffering from von Willebrand's disease type 3, who do not have a detectable von Willebrand Factor in their blood circulation, also suffer from a secondary Factor VIII deficiency. In addition, the half-life of intravenously administered Factor VIII in those patients is 2 to 4 hours, which is considerably shorter than the 10 to 30 hours observed in hemophilia A patients.

vWF not only acts as an extracellular FVIII carrier, but during endothelial FVIII synthesis, vWF also serves as an intracellular chaperone that directs FVIII to releasable storage granules.

One aspect of the present invention is therefore directed to a strategy which facilitates the expression of FVIII in cells, such as megakaryocytes and platelets, where it can interact with vWF.

This was achieved by incorporating a megakaryocyte/platelet specific regulatory region into the nucleic acid construct containing the BDD-FVIII, or other procoagulant, sequence.

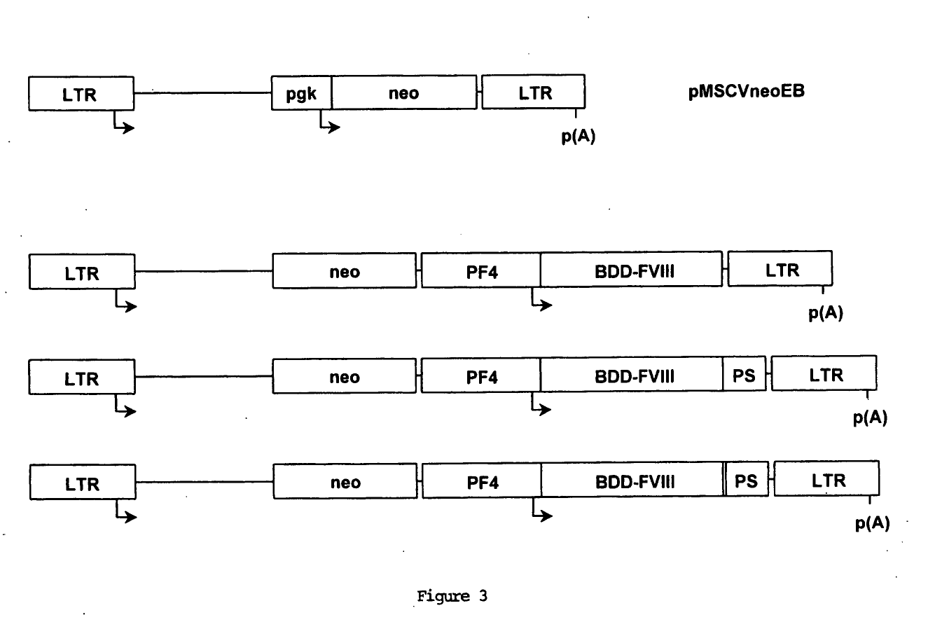

In one exemplary approach, the 1.1 kb 5′ fragment of the rat PF4 gene, which has been shown to confer high level, megakaryocyte-specific reporter gene expression in transgenic mice was obtained (gift of K. Ravid, Boston)⁴ The BDD-FVIII cDNA was placed under the transcriptional control of the PF4 5′ regulatory region by inserting both fragments in tandem, downstream of the neo gene in pBSneo (pBS KSII derivative containing a promoterless neo gene without a polyadenylation signal). From this plasmid backbone, the resultant neo/PF4/BDD-FVIII fusion was shuttled into the retroviral expression construct pMSCVneoEB⁴² (FIG. 3, Panel A) after first removing the existing internal pgk-neo cassette. In the final construct, therefore, neo is under the transcriptional control of the 5′ viral LTR, while the expression of BDD-FVIII is regulated by the PF4 promoter. Both neo and BDD-FVIII polyadenylation signals are supplied by the 3′ viral LTR. The construction of this viral vector is illustrated in FIG. 3, Panel B.

The ability of the resultant vector to direct BDD-FVIII expression in vWF-expressing AtT20 cells was confirmed by confocal microscopy.

Expression in megakaryocytes was also evaluated in vitro using MEG-01, CMK-11-5, and Set-2 cells, which are human megakaryoblastic leukemia cell lines known to express both PF4 and vWF⁴³. Initial lipofectin-transfected, G418-selected clones were screened for BDD-FVIII expression by FVIII-ELISA and/or chromogenic assays of culture supernatants, and by immunofluorescence using polyclonal FVIII antiserum (Dako) and the anti-FVIII monoclonal antibody F-8 respectively.

In parallel, high titre BDD-FVIII-producing retrovirus was prepared in GP+E-86 cells by transfection/selection as above. The viral titre was determined by infection of 3T3 fibroblasts and G418 selection, and the ability of the resultant virus to direct BDD-FVIII expression to megakaryocytes was verified by infection/selection of megakaryocyte cell lines followed by antibody analysis as above. To confirm that BDD-FVIII expression is megakaryocyte-specific, G418 resistant 3T3 fibroblast clones (see above) were analysed in parallel for FVIII expression. Infected megakaryocyte cell lines demonstrate enhanced FVIII production, relative to their 3T3 counterparts, consistent with the tissue-specific effect of the PF4 regulatory elements.

While the description herein has focused on PF4, it is clearly apparent that other platelet specific promoters such as the platelet integrin alpha IIb/GPIIb promoter and other platelet glycoprotein promoters such as the GPVI promoter could also be used within the context of the present invention to achieve tissue specific expression.

It is clearly apparent that other types of vectors may be designed for the targetted delivery of FVIII and other factors. For example, an alternative retrovirus can be constructed using the pMSCVneoEB backbone, in which BDD-FVIII is inserted downstream of the 5′ LTR, the internal pgk-neo cassette is retained, and the enhancer/promoter elements of the U3 region of the 3′ LTR are replaced with the PF4 regulatory elements⁴⁴. After virus generation and infection of target cells, therefore, the reverse-transcribed proviral form of this construct will contain the PF4 regulatory elements in the 5′ LTR such that BDD-FVIII is driven by PF4 sequences, while neo is under the control of the internal pgk promoter. Thus, the PF4 promoter is no longer subject to potential interference from the 5′ LTR.

The present invention demonstrates the ability of the PF4/BDD-FVIII cDNA to target BDD-FVIII expression to megakaryocytes in vivo as well as the ability of endogenous megakaryocyte vWF to act as an intracellular chaperone, thereby directing transgenic BDD-FVIII to platelet α-granules. Specifically, this is done by isolating and infecting murine bone marrow with PF4/BDD-FVIII virus. Following an initial period of drug selection with G418 in vitro to enrich for transduced cells, the marrow is introduced back into lethally irradiated syngeneic animals. This method is known to result in high level, and long term expression of retroviral cDNAs^{27,28}. Following hematopoietic recovery, transplanted animals are examined for megakaryocyte/platelet specific BDD-FVIII expression using standard techniques. Specifically, bone marrow is isolated from transplant recipients and from control animals. Fixed marrow smears are analyzed, for example, by routine Romanowsky staining. BDD-FVIII and vWF can be detected immunocytochemically or by immunofluorescence following cell permeabilization. By dual labelling/immunofluorescence analysis and confocal microscopy it is possible to demonstrate the colocalization of vWF and BDD-FVIII to a-granules, or to the trans-Golgi network in these cells.

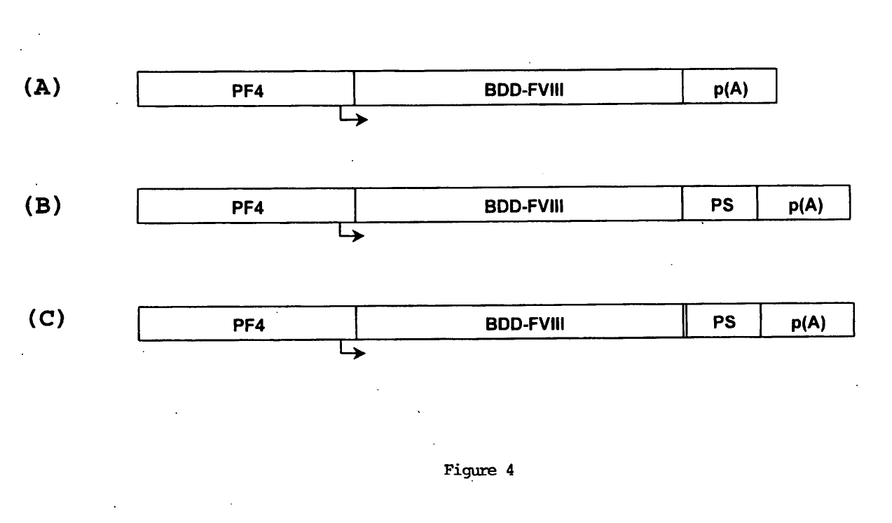

In another aspect of the invention, transgenic mice were prepared by introducing the PF4/BDD-FVIII cDNA by zygote microinjection. The expression construct that was used is illustrated in FIG. 4, Panel A. By this technique, several founders were derived and germline transmission of the transgene was confirmed. The corresponding pedigrees were expanded and several animals were sacrificed and analyzed for transgene expression etc. These animals can be used as bone marrow donors for bone marrow transplantation (BMT) into hemophilic FVIII “Knock-Out” (KO) animals.

The BDD-FVIII targeting strategy described above relies on the intrinsic ability of vWF to act as an intracellular chaperone and to direct BDD-FVIII to α-granules.

The present invention therefore provides means to maximize the amount of BDD-FVIII that is released locally in a regulated fashion following platelet activation by augmenting the targeting of BDD-FVIII to a-granules by other means, both as a backup, and to complement or enhance the vWF effect.

The present invention also encompasses the targeted expression of procoagulant proteins other than, or in addition to, FVIII, and the directed trafficking of those proteins to platelet α-granules. Since vWF targeting is presumably specific to FVIII, an alternative and potentially more generalizable method for directing transgene expression to platelet α-granules is provided.

The sorting of a number of proteins to regulated secretory granules has been shown to be determined by specific targeting domains. For example, the cytoplasmic domain of P-selectin⁴⁸, the COOH tail of the proprotein convertases (PC) PC5-A⁴⁹ and PC1⁵⁰, and the propeptide of preprosomatostatin⁵¹, have been shown to direct the trafficking of a number of proteins to regulated secretory granules. Furthermore, when moved as a module to other proteins, the cytoplasmic domain of P-selectin as well as the preprosomatostatin propeptide confers α-granule targeting to those proteins as well.

In the present invention, the targeting of expression of FVIII and other procoagulant proteins to platelet α-granules by a two-part strategy is disclosed. In a first aspect, the transcription of a BDD-FVIII cDNA, or of another relevant cDNA, is targeted to megakaryocytes using the PF4 5′ promoter or other tissue specific regulatory regions as described above. In a second aspect, the intracellular trafficking of this targeted transgenic protein is directed to α-granules, by incorporating a regulated secretory granule sorting domain, such as the cytoplasmic domain of P-selectin, the COOH tail of the proprotein convertases (PC) PC5-A⁴⁹ and PC1, and the propeptide of preprosomatostatin, into BDD-FVIII as an in-frame fusion.

Prior to the present invention, secretory granule targeting by the cytoplasmic domain of P-selectin has been demonstrated convincingly only for type I transmembrane (TM) proteins (NH2-terminal end is extracellular; COOH-terminal end is cytoplasmic), although this TM domain need not be derived from P-selectin itself. It was not clear how efficiently the P-selectin cytoplasmic domain could target soluble proteins (i.e. without a TM domain) that are normally expressed constitutively, to granules.

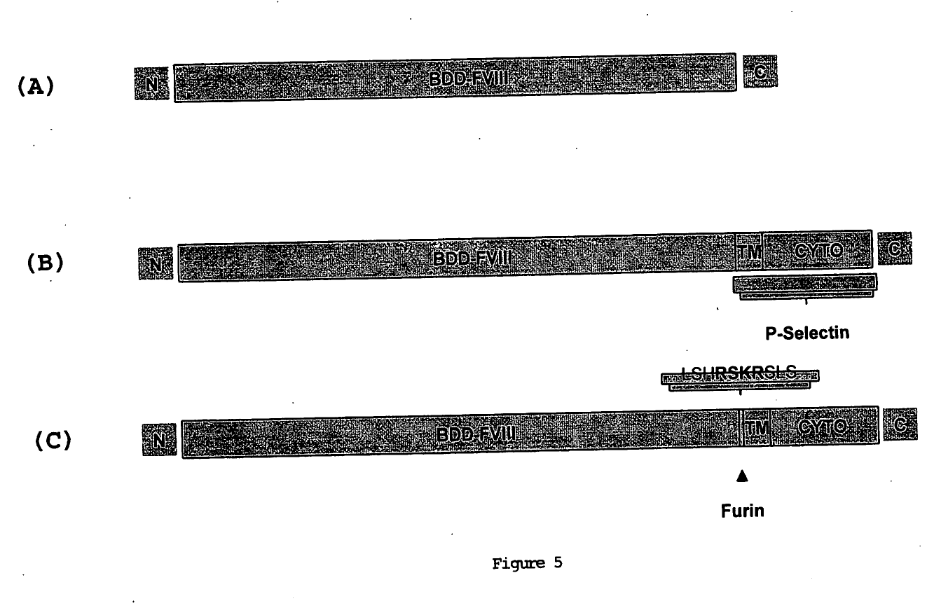

Because the targeting of some soluble proteins may require that they be converted to a membrane bound form by the addition of a TM domain, recombinant PCR was used in the present invention to fuse the sequences encoding the human P-selectin cytoplasmic domain (P-selectin cDNA gift of D. Cutler) with the P-selectin TM domain, to the 3′ end of the BDD-FVIII cDNA, such that the corresponding P-selectin sequences are fused in frame to the COOH- terminus of BDD-FVIII as illustrated in FIG. 5.

While some otherwise soluble procoagulants (e.g. FVIII) may remain functional when tethered to the membrane, this approach was further refined, such that soluble proteins targeted in this fashion would be proteolytically cleaved from their TM anchors once targeting is achieved, thus reverting to a soluble form.

Many eukaryotic protein precursors (or proproteins) are known to undergo limited proteolysis as they transit through intracellular secretory pathways, to yield the mature proteins that are released. Enzymes responsible for this processing comprise the proprotein convertase (PC) family which at present contains seven members, PC1/PC3, PC2, furin/PACE, PC4, PACE4, PC5/PC6, and PC7/SPC7/LPC/PC8 (for review⁵⁵). These enzymes cleave proproteins at specific consensus motifs that fit the general rule—(R/K)-Xₙ-(R/K) (where n=0, 2, 4, or 6, and X can be any amino acid except cysteine)—with each specific PC having a preferred substrate cleavage site motif specificity. As proproteins undergo such processing in transit through secretory pathways, it follows that the PCs specific to each proprotein substrate are targeted in a similar fashion.

While the spectrum of PCs expressed in megakaryocytes has not been defined, the processing of vWF in transit through the megakaryocyte secretory pathways has been studied in detail. Specifically, propolypeptide cleavage of vWF at residue 763 has been localized to the trans-Golgi network (TGN), immediately prior to the formation of the esecretory granule⁵⁶. Since BDD-FVIII, whether it is targeted by the vWF chaperone effect or by engineered targeting domains, must follow an identical TGN to secretory granule route (and in fact associates with vWF prior to granule formation¹¹), it follows that BDD-FVIII colocalize with the PC responsible for the propeptide cleavage of pro-vWF. In vitro studies have demonstrated that there is a specific PC cleavage motif adjacent to vWF residue 763, and that of 3 PCs tested, it is preferentially cleaved by furin/PACE⁵⁶.

Thus, in a further aspect of the present invention, genetic constructs which allow cleavage of soluble BDD-FVIII from the P-selectin targeting domain are provided.

In a preferred embodiment, recombinant PCR was used to construct a BDD-FVIII fusion protein in which the P-selectin targeting domain is separated from the BDD-FVIII COOH-terminus by the pro-vWF propeptide PC cleavage motif described above. This construct is illustrated in FIG. 5, Panel C.

These two P-selectin constructs (with or without the cleavage motif), as well as the original BDD-FVIII cDNA, were inserted into a eukaryotic expression vector, and have also been transfected stably into vWF-expressing AtT-20 cells. Furthermore, transgenes have been micro-injected into mouse zygotes as described above for the PF4/BDD-FVIII. The constructs for generation of transgenic animals are illustrated in FIG. 4. Founders were obtained for the construct that contains the VWF PC cleavage motif, and germline transmission of the transgene has been demonstrated. Amphotropic and ecotropic retroviruses have similarly been constructed and titered for infection of vWF-expressing AtT20 cells and the megakaryocyte cell lines, and for bone marrow transplantation studies, respectively, as described above for the PF4/BDD-FVIII construct (FIG. 3, Panels C and D).

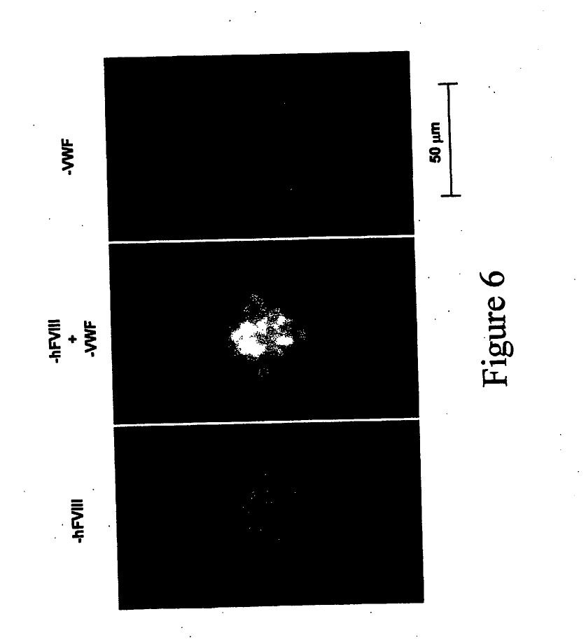

FIG. 6 illustrates that transgenic megakaryocytes express human BDD-FVIII. In one exemplary experiment, bone marrow cells were flushed from the femora of transgenic mice, were counted, and were resuspended at ˜2×10⁶ cells/ml in IMDM supplemented with 2% fetal bovine serum. Cells were then cultured on chamber slides (37° C., 5% CO₂) for 8-10 days in methylcellulose/IMDM containing bovine serum albumin (1%), bovine insulin (10 g/ml), human iron-saturated transferrin (200 g/ml), L-glutamine (2 mM), and 2-mercaptoethanol (10⁻⁴ M)(MegaCult-C; Stem Cell Technologies Inc.), and supplemented with collagen (1.1 mg/ml), rh Thrombopoietin (50 ng/ml), rh IL-6 (20 ng/ml), rh IL-11 (50 ng/ml), and rm IL-3 (10 ng/ml). Resultant megakaryocyte colonies were then dehydrated, fixed with 2% paraformaldehyde, washed, permeabilized with 0.5% Triton/PBS, and stained with murine anti-human FVIII (1:10)(American Diagnostica)/goat anti-mouse IgG-FITC (1:25)(Chemicon), and rabbit anti-human vWF (1:10)(DAKO)/goat anti-rabbit IgG-Rhodamine. Stained cells were then visualized and vWF and FVIII signals were overlayed by confocal immunofluorescence microscopy. In FIG. 6, the expression of human BDD-FVIII (-hFVIII) (left and middle panels) and of von Willebrand Factor (-VWF) (right and middle panels), as assessed by specific immunofluorescent staining, are shown. Transgenic hBDD-FVIII expression colocalizes with that of VWF. The bar indicates 50 μM.

Selected BDD-FVIII expressing cell clones can be analyzed for localization of BDD-FVIII and vWF expression by standard techniques. For example, immunofluorescence can be measured before and after stimulation of regulated granule release with 8-Br-cAMP¹¹. In addition, before and after stimulation, released supernatant BDD-FVIII can be quantified and tested functionally by a commercial BDD-FVIII-ELISA and chromogenic assay, respectively. Cell surface BDD-FVIII can also be evaluated by standard immunofluorescence techniques, and function can be assessed by modifying the BDD-FVIII:C assay for use on cell monolayers.

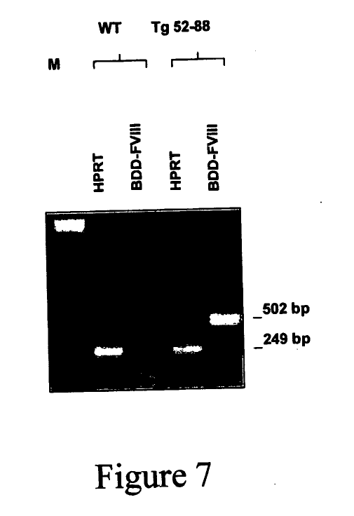

FIG. 7 illustrates that human BDD-FVIII RNA is expressed by transgenic bone marrow cells. In an exemplary experiment, bone marrow cells were flushed from the hind limbs of WT and transgenic animals, and total RNA was extracted. After DNAse treatment of 5 g of RNA, cDNA was prepared using the random priming method. PCR was then carried out with 1 I cDNA (1/20 of the total cDNA synthesis reaction) using the human BDD-FVIII specific oligonucleotides 5′-GCACAGACTGACTTCCTTTC-3′ and 5′-GGCTCTGATTTTCATCCTCA-3′ which yield a 523 bp product, and the murine HPRT specific oligonucleotides 5′-GCTGGTGAAAAGGACCTCT-3′ and 5′-CACAGGACTAGAACACCTGC-3′, which yield a 249 bp product. PCR products were size-separated electrophoretically and visualized following ethidium bromide staining.

FIG. 7 illustrates the results obtained when RT-PCR was used to assess the expression of human BDD-FVIII by transgenic (Tg 52-88) and non-transgenic (WT) bone marrow cells. While transgenic bone marrow yielded a 523 bp human BDD-FVIII specific PCR product, WT bone marrow did not. In contrast, both samples produced 249 bp signals specific to the housekeeping gene hypoxanthine phophoribosyl transferase (HPRT). Control reactions performed without reverse transcription did not yield any bands (not shown). M, DNA size markers.

Transgenic mice expressing the PF4/BDD-FVIII/targeting domain fusion proteins can be used in standard bone marrow transplantation techniques as described above for the basic PF4/BDD-FVIII construct.

The genetic constructs of the present invention provide agents for the gene therapy of Hemophilia A. The clinical efficacy of the constructs can be assessed using standard gene therapy techniques well known to those skilled in the art. For example, the retroviral targeting constructs (using either the vWF chaperone or the targeting domain fusion protein strategy) can be evaluated for clinical efficacy in FVIII-deficient mice in which the FVIII gene has been inactivated by homologous recombination-mediated gene targeting in embryonic stem cells²³⁻²⁶. Bone marrow can be infected with the appropriate retrovirus and then re-infused into lethally irradiated FVIII-/-recipients, according to well-established methods. Targeted protein expression can be assessed at various times post transplant (e.g. 6 weeks, 4 months, 8 months, 12 months) using standard techniques.

Local levels of FVIII following platelet activation at sites of vascular injury can also be assessed and functional activity determined using well-known assays. For example, tail bleeding time and rate of blood flow can be assayed following standardized transection of the tail tip^{23,25,57} in anaesthetized transplanted animals and in untransplanted controls, beginning at 6 weeks after transplant.

The techniques established using the murine models can be extended to human patients for the treatment of disease.

The present invention has several advantages over other gene therapy approaches for Hemophilia. FVIII and/or other proteins targeted by this approach accumulate within α-granules, and are therefore available for regulated local release following platelet activation at sites of injury. The procoagulant activity is targeted not only to areas of vascular injury, but also to sites at which secondary rebleeding occurs. Furthermore, since the targeted protein is sequestered in α-granules and is not released until platelet activation, even low levels of constitutive transgenic protein expression will result in high local FVIII levels at the sites of bleeding. Thus, the approach is safe, efficacious and durable.

There are also several immunological advantages associated with the present invention. Since bone-marrow mediated exposure to antigen is generally less immunogenic than is parenteral exposure to the same antigen, the bone marrow transplantation methods of the present invention should reduce the formation of FVIII or of other protein inhibitors, and may induce tolerance in those with pre-existing inhibitors. Furthermore, the targeting of natural procoagulants, such as hepsin, according to the methods of the present invention, is likely not to be as immunogenic as is the expression of FVIII in a hemophilic background.

Although preferred embodiments of the invention have been described herein in detail, it will be understood by those skilled in the art that variations may be made thereto without departing from the spirit of the invention and the scope of the appended claims.

### References

1. Luchtman-Jones, L. & Broze Jr., G. Ann. Med. 27, 47-52 (1995).

2. Broze Jr., G. Blood Coag. Fibrinolys. 6, S7-S13 (1995).

3. Osterud, B. Blood Coag. Fibrinolys. 6, S20-S25 (1995).

4. Conway, E. M., Bach, R., Rosenberg, R. D. & Konigsberg, W. H. Thromb. Research 53, 231-241 (1989).

5. Wada, H., Wakita, Y. & Shiku, H. Blood Coag. Fibrinolys. 6, S26-S31 (1995).

6. Rottingen, J., Emden, T., Camerer, E., Iversen, J. & Prydz, H. J. Biol Chem. 270, 4650-4660 (1995).

7. Rapaport, S. & Rao, L. Arterioscler. Thromb. 12, 1111-1121 (1992).

8. Sadler, J. & Davie, E. in The molecular basis of blood disease (ed. Stamatoyannopoulos, N., Majerus, Varmus) 657-700 (W. B. Saunders, Philadelphia, 1994).

9. Kaufman, R. Ann. Rev. Med. 43, 325 (1992).

10. Wagner, D. Ann. Review Cell. Biol. 6, 217 (1990).

11. Rosenberg, J., et al. J. Clin. Invest. 101, 613-624 (1998).

12. Wilcox D A, Rosenberg J F, Johnson B D, Montgomery R R. Blood (Suppl) In Press (2000).

13. Ginsburg, D., Roberts, H. & High, K. Blood suppl., 2947 (1997).

14. Brackman, H., Eickhoff, H., Oldenburg, J. & Hammerstein, U. Haemost. 22, 251-258 (1992).

15. Hilgartner, M., Manno, C., Nuss, R. & DiMichele, D. Blood suppl., 46-60 (1997).

16. Roberts, H. N. EngL. J. Med. 321, 1188-1189 (1989).

17. Nilsson, I., Berntorp, E. & Zettervall, 0. N. Engl. J. Med. 318, 947 (1988).

18. Lusher, J., et al. Blood 62, 1135-1139 (1983).

19. Hedner, U., Glazer, S. & Falch, J. Transfusion Med 7, 78-83 (1993).

20. Nicolaisen, E., Hansen, L., Poulsen, F., Glazer, S. & Hedner, U. Thromb. Haemost. 76, 200-204 (1996).

21. Pittman, D., et al. Blood 81, 2925-2935 (1993).

22. Berntorp, E. Thromb. Haemsot. 78, 261 (1997).

23. Bi, L., et al. Nat. Genet. 69, 21-24 (1995).

24. Bi, B., etal. Blood 88, 3446-3450 (1996).

25. Turecek, P., et al. Thromb. Haemost. 77, 591-599 (1997).

26. Fakharzadeh, S., Sarkar, R. & Kazazian Jr., H. Thromb. Haemost. suppl., (1997).

27. Dwarki, V., et al. Proc. Natl. Acad. Sci. (USA) 92, 1023-1027 (1995).

28. Chuah, M., Vandendriessche, T. & Morgan, R. Hum. Gene Ther. 6, 1363-1377 (1995).

29. Vandendriessche T, Vanslembrouck V, Goovaerts I, Zwinnen H, Vanderhaeghen ML, Collen D, Chuah M. Proc. Natl. Acad. Sci. (USA) 96, 10379-10384 (1999).

30. Zatloukal, K., et al. Proc. Natl. Acad. Sci. (USA) 91, 5148-5152 (1994).

31. Connelly, S., Gardner, J., McClelland, A. & Kaleko, M. Hum. Gene Ther. 7,183-195 (1996).

32. Connelly, S., Andrews J L, Gallo AM, Kayda D B, et al. Blood 9, 3273-3281 (1998).

33. Connelly, S., et al. Blood 88, 3846-3853 (1996).

34. Gao, G., Yang, Y. & Wilson, J. J. Virol. 70, 8934-8943 (1996).

35. Qian, J., Scott, D. & Hoyer, L. Thromb. Haemost. suppl., (1997).

36. Naldini, L., et al. Science 272, 263-267 (1996).

37. Gnatenki, D., Hearing, P., Gergel, J., Jesty, J. & Bahou, W. Blood 88, 139 (1996).

38. III, C., et al. Thromb. Haemost. suppl., (1997).

39. Mohri, H., Tanabe, J., Ohtsuka, M., Yoshida, M., Motomura, S., Nishida, S., Fujimura, Y., & Okubo, T. Blood Coag. Fibrinolys. 6:561-166 (1995).

40. Evans, G. & Morgan, R. Blood 90, 1053a (1997).

41. Ravid, K., Beeler, D. L., Rabin, M. S., Ruley, H. E. & Rosenberg, R.D. Proc Natl Acad Sci Usa 88, 1521-1525 (1991).

42. Hawley, R., et al. Proc. Natl. Acad. Sci. (USA) 93, 10297-10302 (1996).

43. Hassan, H. & Freund, M. Leuk. Res. 19, 589-594 (1995).

44. Diaz, R., Eisen, T., Hart, I. & Vile, R. J. Virol. 72, 789-795 (1998).

45. Hawley, R., Covarrubias, L., Hawley, T. & Mintz, B. Proc. Natl. Acad. Sci. (USA) 84, 2406-2410 (1987).

46. Hawley, R., Lieu, F., Fong, A. & Hawley, T. Gene Therapy 1, 136-138 (1994).

47. Shalaby, R., Rossant, J., Yamaguchi, T., Breitman, M. & Schuh, A. Nature (1995).

48. Disdier, M., Morrissey, J., Fugate, R., Bainton, D. & McEver, R. Mol. Biol. Cell 3, 309-321 (1992).

49. De Bie, I., et al. J. Cell Biol. 135, 1261-1275 (1995).

50. Seidah, N., personal communication (1998).

51. Stoller, T. & Shields, D. J. Cell Biol. 108, 1647-1655 (1989).

52. Hartwell, D., et al. Blood 90 Suppl. 1, 567a (1997).

53. Ishiwata, N., et al. J. Biol. Chem. 269, 23708-23705 (1994).

54. Sporn, L. A., Marder, V. J. & Wagner, D. D. Cell 46, 185-190 (1986).

55. Seidah, N. & Chretien, M. Curr. Opin. Biotech. 8, 602-607 (1997).

56. Rehemtulla, A. & Kaufman, R. Blood 79, 2349-2355 (1992).

57. Wu Q., Yu, D., Post, J., Sadler, J. & Morser, J. Thromb. Haemost. 78 (suppl), PD3090 (1997).

1. A nucleic acid sequence comprising all or part of a gene sequence encoding a procoagulant factor operably linked to a megakaryocyte/platelet specific regulatory region.

2. A nucleic acid sequence according to claim 1 further comprising a secretory granule-sorting domain.

3. A nucleic acid sequence according to claim 1 wherein the procoagulant factor is Factor VIII.

4. A nucleic acid sequence according to claim 1 wherein the procoagulant factor is hepsin.

5. A nucleic acid sequence according to claim 1 wherein the megakaryocyte/platelet specific regulatory region is selected from the group consisting of the PF4 promoter, the platelet integrin alpha IIb/GPIIb promoter, the GPVI promoter and other platelet glycoprotein promoters.

6. A nucleic acid sequence according to claim 2 wherein the secretory granule sorting domain is selected from the group consisting of the cytoplasmic domain of P-selectin and the carboxy-terminal tails of the proprotein convertases PC5A and PC1.

7. A nucleic acid sequence encoding amino acids 1-740 and 1649-2351 of human Factor VIII joined by a linking fragment comprising residues 741-760 and 1631-1648 of human Factor VIII.

8. A B-domain deleted form of Factor VIII wherein residues 761-1630 of human Factor VIII have been deleted.

9. A vector for expression of the nucleic acid sequence defined in claim 1.

10. A vector according to claim 9 wherein the vector is a retroviral vector.

11. A genetically modified cell expressing the nucleic acid sequence defined in claim 1.

12. A transgenic animal expressing the nucleic acid sequence defined in claim 1.

13. A method of treating hemophilia A, said method comprising the steps of:
i) providing a nucleic acid construct comprising a sequence encoding a procoagulant factor operably linked to a tissue-specific promoter;
ii) introducing the nucleic acid construct into bone marrow cells to obtain genetically modified cells; and
iii) implanting said genetically modified cells into a patient.

14. (Canceled)

15. A method of gene therapy, said method comprising administering to a patient in need thereof a therapeutically effective amount of a viral vector comprising a nucleic acid sequence encoding a Factor VIII gene product, wherein expression of the Factor VIII gene product is regulated by a megakaryocyte specific promoter.

16. (Canceled)

17. A method of treating hemophilia A, said method comprising administering to a patient in need thereof an effective amount of a nucleic acid of claim 1.

18. A method of treating hemophilia A, said method comprising administering to a patient in need thereof an effective amount of a nucleic acid of claim 2.

19. A method of treating hemophilia A, said method comprising administering to a patient in need thereof an effective amount of a nucleic acid of claim 3.

20. A method of treating hemophilia A, said method comprising administering to a patient in need thereof an effective amount of a nucleic acid of claim 4.

21. A method of treating hemophilia A, said method comprising administering to a patient in need thereof an effective amount of a nucleic acid of claim 5.

22. A method of treating hemophilia A, said method comprising administering to a patient in need thereof an effective amount of a nucleic acid of claim 6.
